(19) United States
(12) Patent Application Publication
(10) Pub. No.: US 2011/0065836 A1
(43) Pub. Date: Mar. 17, 2011

(54) POLYCOSANOL-BASED ASSOCIATIVE MONOMERS, CORRESPONDING ASSOCIATIVE THICKENING AGENTS AND THEIR USES

(75) Inventors: Jean-Marc Suau, Lucenay (FR); Denis Ruhlmann, Genay (FR)

(73) Assignee: COATEX S.A.S., Genay (FR)

(21) Appl. No.: 12/843,963

(22) Filed: Jul. 27, 2010

Related U.S. Application Data
(60) Provisional application No. 61/247,004, filed on Sep. 30, 2009.

(30) Foreign Application Priority Data
Sep. 11, 2009 (FR) 09 04335

(51) Int. Cl. C08L 63/00; C07D 303/04; C08F 224/00

(52) U.S. Cl. 523/400; 549/512; 526/273

(57) ABSTRACT

The present invention relates to new associative monomers, terminated by a hydrophobic chain with a base of polycosanols, which can be bio-resourced raw materials, and notably of octacosanol. It also concerns the HASE-type associative copolymers manufactured from these monomers, from (meth)acrylic acid and from an ester of one of these acids. The invention also concerns the use of these copolymers as thickening agents for water-based formulations.

### REFERENCE TO PRIOR APPLICATIONS

This application claims priority to U.S. provisional application 61/247,004 filed Sep. 30, 2009, and to French patent application 09 04335 filed Sep. 11, 2009, both incorporated herein by reference.

### FIELD OF THE INVENTION

The present invention concerns the sector of associative thickening agents, and more specifically of associative thickening agents of the HASE (Hydrophobically modified Alkali-Soluble Emulsions) type. These products can be used in water-soluble formulations such as paints.

The present invention also concerns new associative monomers based on polycosanols, which can be a bio-resourced raw material. These monomers can be used in the manufacture of HASE-type thickening agents, thus providing a new range of rheological additives optionally of bio-resourced origin. In addition, the thickening agents give water-based formulations into which they are introduced very advantageous properties, such as thixotropic effects.

In preferred embodiments the present invention relates to new associative monomers terminated by a hydrophobic chain with a base of polycosanols, and notably of octacosanol. It also concerns the HASE-type associative copolymers manufactured from these monomers, from (meth)acrylic acid and from an ester of one of these acids.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In describing the background of the invention it is the intention of the inventors to prime the reader for a greater understanding of the invention. In doing so certain documents, patents, etc. may be described. These documents speak for themselves, and although any discussion of their contents herein is believed to be accurate, any inadvertent mischaracterization does not constitute an admission.

Water-soluble paint formulations containing mineral fillers consist of an aqueous phase, one or more polymers in emulsion in the liquid phase called binders, fillers and/or pigments, a dispersing agent and additives as diverse as surfactants, coalescence agents, biocides, anti-foaming agents and, finally, at least one thickening agent.

The latter enables the rheology of the water-soluble formulations into which it is introduced, and notably water-soluble paints, to be controlled, both at the manufacturing stage, and during transport or storage, or at the time of application. The wide variety of practical constraints in each of these stages relates to a multiplicity of different rheological properties. Nevertheless, it is possible to summarise the requirement of the skilled man in the art in obtaining an effect of the thickening of the water-based formulation, both for reasons of stability over time, and for a possible application of the paint to a vertical surface, lack of splashing during use, etc. For this reason the additives which contribute to this regulation of the rheological properties have been designated using the term thickening agents.

Among these products, thickening agents known as “associative” thickening agents are distinguished, which are water-soluble polymers having insoluble hydrophobic groups. Such macromolecules have an associating character: when introduced into water, hydrophobic groupings tend to assemble in the form of micellar aggregates. These aggregates are linked together by the hydrophilic parts of the polymers: there is then formation of a three-dimensional network which causes the viscosity of the medium to be increased.

The action mechanism and the characteristics of the associative thickening agents are now well known and described, for example in the documents “Rheology modifiers for water-borne paints” (Surface Coatings Australia, 1985, pp. 6-10) and “Rheological modifiers for water-based paints: the most flexible tools for your formulations” (Eurocoat 97, UATCM, vol. 1, pp 423-442).

Among these associative thickening agents, the class of associative thickening agents of the HASE (Hydrophobically modified Alkali-soluble Emulsions) type is distinguished. They designate copolymers of (meth)acrylic acid, of an ester of these acids, and of a “hydrophobic” monomer, which in fact consists of an oxyalkylated chain which is functionalisable at one end and terminated by a hydrophobic group at the other end. The choice of the terminal hydrophobic group is particularly important, since to a large extent it determines the rheological properties induced by the corresponding thickening agent.

The documents “Dynamic light scattering of semi-dilute hydrophobically modified HASE solutions with varying length of hydrophobic alkyl chains” (Macromol. Chem. Phys., 203, 2002, pp. 2312-2321), and “Light scattering of dilute HASE solutions: effects of hydrophobicity and spacer length of macromonomer” (Macromolecules, 33, 2000, pp. 7021-7028) show that the low-shearing gradient viscosity of a water-based paint containing thickening agents of the HASE type increases, if the number of carbon atoms in the hydrophobic group is greater than 16.

The document “Viscoelastic properties of HASE emulsion in salt solutions” (Polymer 40, 1999, pp. 6369-6379), which relates to HASE-type thickening agents, the hydrophobic group of which is an alkyl chain having 8 to 20 carbon atoms, shows that whatever the shearing gradient considered, the viscosity increases in a very substantial manner with the number of carbon atoms borne by the alkyl chain.

Thus, the overall teaching provided by these documents is that in order to obtain greater thickening efficiency one solution consists in increasing the number of carbon atoms borne by the hydrophobic grouping. This is one of the elements of general knowledge of the skilled man in the art: for a given type of hydrophobic grouping, if the number of carbon atoms is increased, the viscosity of the water-based formulation will be increased. Indeed, the size of the hydrophobic groupings will determine the size of the micellar aggregates which they engender once in solution, which is directly related to an increase of the viscosity, as is recalled by the document “Rheology modifiers for water-borne paints” (Surface Coatings Australia, 1985, pp. 6-10).

This teaching is corroborated by a number of patents which describe and claim, for HASE-type polymers, terminal hydrophobic groups the number of carbon atoms of which may be equal to 30 and, in a very particular case described below, greater than 40.

Thus, document EP 0 013 836 reveals a copolymer with a methacrylic acid and alkyl acrylate base having up to 4 carbon atoms and an oxyalkylated monomer terminated by a fatty chain having up to 30 carbon atoms, capable of substantially increasing the Brookfield viscosity of water-based gels.

Document EP 0 011 806 describes liquid polymers in emulsion used as thickening agents for water-based compositions—and notably latex paints—resulting from the copolymerisation of a carboxylic acid-based acid monomer, a monomer which is ethyl acrylate or blends of it with another monomer, and finally a non-ionic vinylic surface active ester terminated by an alkyl chain having 8 to 20 carbon atoms, or a phenyl alkyl chain having 8 to 16 carbon atoms. These polymers prove efficient at medium and high shearing gradients, as shown respectively by the Stormer™ viscosity measurements (medium shearing gradient) and Haake™ viscosity measurements (high shearing gradient equal to 10,000 s⁻¹) taken for this type of polymer.

Document EP 0 013 836 teaches the existence of copolymers obtained by copolymerisation in emulsion of acrylic or methacrylic acid, of an alkyl methacrylate or acrylate, and of a non-ionic monomer terminated by an alkyl grouping, having 8 to 30 carbon atoms. Such additives enable the storage properties of water-based formulations to be improved, i.e. their low shearing gradient viscosity to be increased, as the values of the Brookfield™ viscosities measured at 10 rpm indicate.

Document EP 0 577 526 describes copolymers which are obtained by copolymerisation of an ethylenic monomer with a carboxylic function, possibly a monomer with ethylenic unsaturation and without a carboxylic function, and an oxyalkylate monomer with ethylenic unsaturation terminated by a hydrophobic fatty chain, itself constituted of linear or branched groups of the alkyl, alkylaryl, arylalkyl or aryl type, having 26 to 30 carbon atoms. Such thickening agents prove to be particularly effective for developing high viscosities with low shearing gradients in water-based formulations.

Similarly, document EP 1 270 617 describes copolymers of identical structure, which give water-based formulations a similar rheological profile, the polymers having a fatty chain consisting of linear or branched alkyl, alkylaryl, arylalkyl or aryl groups which may have up to 36 carbon atoms.

At the same time, document EP 0 705 852 describes a rheological additive which is a copolymer obtained by copolymerisation of one or more carboxylic acid monomers, one or more non-ionic vinylic monomers, one or more non-ionic monomers containing a hydrophobic phenol grouping substituted by an arylkyl, such as tristyrylphenol, which has 30 carbon atoms. (The Applicant states that this compound is already well known for its surface active properties, as is demonstrated by document WO 97/03242, which describes a composition enabling UV rays to be absorbed, notably containing di- and tristyrylphenols as surfactants). Such thickening agents give water-based paints satisfactory rheological properties with a high and medium speed gradient.

Finally, document EP 1 812 482 describes a process for manufacturing derivatives of cumyl phenol having more than 40 carbon atoms, and in certain cases more than 50 carbon atoms: these products are used in the composition of HASE-type associative thickening agents in their hydrophobic group.

In the light of the prior art, the number of carbon atoms borne by the hydrophobic group with a view to obtaining “effective” thickening properties can be quantified at “at least 16”, preferentially “at least 20”, and very preferentially “at least 24”. In addition to this technical requirement, there is a constraint of an environmental nature: that of currently obtaining bio-resourced products, i.e. those not produced from a fossil energy source. This approach is one aspect of the concept of green chemistry and sustainable development.

With this regard, science has unfortunately made little progress: although stearine (18 carbon atoms) has had a certain success, in the context of hydrophobic groups having more than 24 carbon atoms, only cholesterol and lanolin have, to our knowledge, been envisaged (respectively 27 and 30 carbon atoms), but anecdotally (see page 15 of document EP 0 013 836). Consequently, there is currently great demand for HASE-type associative thickening agents, having a terminal hydrophobic group having at least 24 carbon atoms, and of bio-resourced origin.

### BRIEF DESCRIPTION OF THE DRAWING

FIG. 1 represents the rheograms obtained for a non-thixotropic thickening agent which is Viscoatex 730 (shown by the dotted grey line), for a polymer of the prior art A6 (grey line) and for a polymer according to the invention C-5 (black line).

### DETAILED DESCRIPTION OF THE PREFERRED EMBODIMENTS

The inventors have succeeded in developing new structures of the type described above. They have notably succeeded in developing new hydrophobic monomers, which can be used in the synthesis of HASE-type associative thickening agents, where the monomers have the following formula (I):

R-(A-O)ₘ—(B—O)ₙ—R′ (I)

where:
m and n are independently integers of less than 150, at least one of which is non-zero,
A and B designate alkyl groups which are different one from another, and having 2 to 4 carbon atoms, where group AO preferentially designates ethylene oxide, and group BO preferentially designates propylene oxide,
R designates a polymerisable unsaturated group, preferentially methacrylate,
where R′ is a grouping of formula (II):

CH₃—(CH₂)ₚ—CH₂— (II)

where p is a real number of between 24 and 34, preferentially equal to 26 (in this latter case R′ designates octacosanol).

This latter formula can be obtained from polycosanyls or derivatives of polycosanols, the latter being fatty alcohols derived from sugar cane, beeswax and rice polishings. The most widely used polycosanols are octacosanol, triacontanol, dotriacontanol, hexacosanol and heptacosanol. The formula of octacosanol, a preferred constituent, is CH₃—(CH₂)₂₆—CH₂—OH. These products are known today for their therapeutic efficacy: cholesterol-lowering property, improvement of the lipid status, reduced platelet aggregation, reduced proliferation of muscular cells, and improvement of cardiovascular symptoms (see notably documents EP 2 007 429, EP 1 827 136, EP 1 536 693, EP 1 776 159 and EP 1 515 714).

Nothing in the state of the technique suggested that such structure might be used to manufacture hydrophobic monomers, used for example in the composition of HASE-type associative thickening agents. By this means a non-polluting raw material, which is easily available in large quantities, is exploited.

In addition, it transpires that the thickening agents of the invention lead to particularly advantageous rheological effects, when used in water-based compositions. As is demonstrated by the tests below which illustrate, but do not limit, the present invention, a thickening effect is obtained over a wide range of shearing gradients, depending on the synthesised structure: it is therefore now possible to offer the end user a genuine range of products. Finally, pigmentary compatibility is not damaged, and it is possible to now obtain particularly advantageous thixotropy effects with a view to improving application compromises of the drip flow type.

Ultimately, therefore, this is a particularly effective HASE-type thickening agent within the water-based compositions into which it is introduced, which has an associative monomer terminated by a hydrophobic group having more than 20 carbon atoms (24 to 34 preferably), where the hydrophobic group may be of entirely bio-resourced origin.

Thus a first object of the invention is hydrophobic monomers of formula (I):

R-(A-O)ₘ—(B—O)ₙ—R′ (I)

where:
m and n are independently integers of less than 150, at least one of which is non-zero,
A and B designate alkyl groups which are different one from another, and having 2 to 4 carbon atoms, where group AO preferentially designates ethylene oxide, and group BO preferentially designates propylene oxide,
R designates a polymerisable unsaturated group, preferentially methacrylate,
where R′ is a grouping of formula (II):

CH₃—(CH₂)ₚ—CH₂— (II)

where p is a real number between 24 and 34, preferentially equal to 26.

A second object of the present invention is water-soluble copolymers comprising, consisting essentially of, and consisting of monomer units derived from:
a) (meth)acrylic acid,
b) at least one ester of (meth)acrylic acid,
c) of at least one monomer of formula (I) as described above.

The manufacture of these copolymers, which belong to the family of HASE-type thickening agents, is well within the ability of the skilled man in the art, who can refer to the teaching of the documents cited above as technological background of the present invention.

Preferably these copolymers comprise, expressed as a % by weight of each of the monomers, where the sum of a), b) and c) is preferably equal to 100%:
a) 20% to 50%, preferentially 35% to 45%, of (meth)acrylic acid,
b) 40% to 70%, preferentially 45% to 55%, of at least one ester of (meth)acrylic acid,
c) 2% to 20%, preferentially 3% to 15%, of at least one monomer of formula (I).
   Preferred embodiments include:

A hydrosoluble copolymer comprising monomer units derived from a), b), and c) in the following amounts by weight based on total weight:

a) 20% to 50% of (meth)acrylic acid,

b) 40% to 70% of at least one ester of (meth)acrylic acid, and

c) 2% to 20% of at least one monomer of formula (I);

A hydrosoluble copolymer comprising monomer units derived from a), b), and c) in the following amounts by weight based on total weight:

a) 35% to 45% of (meth)acrylic acid,

b) 45% to 55% of at least one ester of (meth)acrylic acid, and

c) 3% to 15% of at least one monomer of formula (I).

Examples of esters of (meth)acrylic acid useful herein include, e.g., C₂-C₈ (meth)acrylates such as ethyl acrylate, ethyl methacrylate, etc, the term (meth) denoting the presence or absence of this prefix in all occurrences herein.

The invention copolymers may optionally comprise other monomers copolymerizable with those of a), b) and c) for example in amounts of up to 40% and more based on total weight, for example 5%, 10%, etc., and may be obtained for example by radical polymerisation in solution, in a direct or reverse emulsion, in suspension or precipitation in solvents, in the presence of catalytic systems and transfer agents, or again under controlled radical polymerisation, and preferentially under nitroxide mediated polymerisation (NMP) or by cobaloximes, under atom transfer radical polymerisation (ATRP), under controlled radical polymerisation by sulphurated derivatives, chosen from among carbamates, dithioesters or trithiocarbonates (RAFT) or xanthates.

A third object of the present invention is the use of the described water-soluble copolymers as thickening agents in a water-based composition, where the composition is preferentially chosen from among a water-based paint, a lacquer, a varnish, a paper coating, or a cosmetic or detergent formulation.

The following examples further describe the invention, allowing it to be better understood, without however limiting its scope.

### EXAMPLES

### Example 1

This example describes 5 water-soluble copolymers, using new octacosanol-based hydrophobic monomers. These copolymers will be subject to various uses in the following examples.

Test n^{o} 1

This test corresponds to a water-soluble copolymer, expressed as a % by weight of each of its constituents:
a) 36.4% methacrylic acid,
b) 53.2% of ethyl acrylate,
c) 10.4% of a monomer of formula (I):

R-(A-O)ₘ—(B—O)ₙ—R′ (I)

where m=25, n=0 and AO designates ethylene oxide, R is the methacrylate function and R′ designates octacosanol.
Test n^{o} 2

This test corresponds to a water-soluble copolymer, expressed as a % by weight of each of its constituents:
a) 36.4% methacrylic acid,
b) 53.2% of ethyl acrylate,
c) 10.4% of a monomer of formula (I):

R-(A-O)ₘ—(B—O)ₙ—R′ (I)

where m=50, n=0 and AO designates ethylene oxide, R is the methacrylate function and R′ designates octacosanol.
Test n^{o} 3

This test corresponds to a water-soluble copolymer, expressed as a % by weight of each of its constituents:
a) 39.3% methacrylic acid,
b) 55.7% of ethyl acrylate,
c) 5.0% of a monomer of formula (I):

R-(A-O)ₘ—(B—O)ₙ—R′ (I)

where m=25, n=0 and AO designates ethylene oxide, R is the methacrylate function and R′ designates octacosanol.
Test n^{o} 4

This test corresponds to a water-soluble copolymer, expressed as a % by weight of each of its constituents:
a) 39.3% methacrylic acid,
b) 55.7% of ethyl acrylate,
c) 5.0% of a monomer of formula (I):

R-(A-O)ₘ—(B—O)ₙ—R′ (I)

where m=50, n=0 and AO designates ethylene oxide, R is the methacrylate function and R′ designates octacosanol.
Test n^{o} 5

This test corresponds to a water-soluble copolymer, expressed as a % by weight of each of its constituents:
a) 40.5% methacrylic acid,
b) 56.0% of ethyl acrylate,
c) 2.5% of a monomer of formula (I):

R-(A-O)ₘ—(B—O)ₙ—R′ (I)

where m=25, n=0 and AO designates ethylene oxide, R is the methacrylate function and R′ designates octacosanol.

### Example 2

This example illustrates the use of copolymers according to the invention and the prior art, as thickening agents of a solvent-free matt paint.

The composition of the paint is given in table 1, where the masses of each constituent are given in grams. All the thickening agents have a dry extract of 30% by weight of active matter. The paint is formulated using the methods well known to the skilled man in the art.

TABLE 1

| Ingredients of the water-based paint formulation | Masses (g) |
|---|---|
| Ecodis ™ P50 (COATEX ™ dispersing agent) | 4.1 |
| Tego 1488 ™ (TEGO ™ anti-foaming agent) | 2.3 |
| Mergal K6N (TROY ™ bactericide) | 2.3 |
| TiONa RL68 (TiO₂ MILLENIUM ™) | 90 |
| Omyacoat ™ 850 OG (CaCO₃ OMYA ™) | 223 |
| Durcal ™ 5 (CaCO₃ OMYA ™) | 272 |
| Mowilith ™ LDM 1871 (CLARIANT ™ binder) | 180 |
| NaOH | 2.6 |
| Tested copolymer | 5.0 |
| Water | 218.7 |
| Total | 1000 |

The resulting viscosities are then determined at various speed gradients:
at low gradient: Brookfield™ viscosity at 10 and 100 revolutions per minute, respectively noted μ_{Bk 10} and μ_{Bk 100} (mPa·s),
at medium gradient: Stormer viscosity, noted μ_{S}
at high gradient: ICI viscosity, noted μ₁
   at times T=0, 24 hours and 7 days.

It is recalled that in the field of water-based paints a high viscosity with high shearing gradient reflects “dynamic” properties: in practice the viscosity of the paint remains sufficiently high during the stage of application on to the support; its benefits may be a greater high build (i.e. deposited thickness), and a reduced propensity to splashing. At the same time, a high viscosity with a low or medium shearing reflects “static” properties: satisfactory stability is ensured during their storage, whilst preventing the phenomenon of sedimentation and limitation of the tendency to drip on a vertical support.

Tests A1, A2, A3, A4 use respectively copolymers 1, 2, 3 and 4 according to the invention (INV) and test A6 uses a copolymer consisting, by weight, of 55% of ethyl acrylate, 37% of methacrylic acid and 8% of a monomer of formula (I), where m=25, n=0, R is the methacrylate group and R′ designates a branched hydrocarbonated chain containing 32 carbon atoms and derived from petrochemical processes. The results are shown in table 2.

TABLE 2

| | | test A1 | test A2 | test A3 | test A4 | test A6 |
|---|---|---|---|---|---|---|
| | | INV | INV | INV | INV | PA |
| μ_{B10}/μ_{B100} | T = 0 | 7800/1000 | 8300/1200 | 10500/1400 | 10800/1500 | 14900/1900 |
| μ_{S} | | 73 | 75 | 79 | 81 | 84 |
| μ_{I} | | 0.4 | 0.4 | 0.4 | 0.5 | 0.5 |
| μ_{B10}/μ_{B100} | T = 24 hours | 12000/1600 | 13100/1700 | 15600/2000 | 15000/1950 | 18500/2200 |
| μ_{S} | | 86 | 89 | 92 | 91 | 95 |
| μ_{I} | | 0.4 | 0.4 | 0.4 | 0.5 | 0.5 |
| μ_{B10}/μ_{B100} | T = 7 days | 15000/2000 | 15500/1200 | 18000/3200 | 17200/2250 | 20000/2500 |
| μ_{S} | | 92 | 93 | 96 | 95 | 98 |
| μ_{I} | | 0.4 | 0.4 | 0.4 | 0.5 | 0.5 |

This table demonstrates that the copolymers according to the invention enable a solvent-free matt paint to be thickened effectively, whatever the shearing gradient. Although the viscosities obtained are close to those measured for reference, the variations observed depending on the synthesised structures demonstrate the ability to develop a range of thickening agents from such copolymers. The latter therefore constitute an effective alternative in terms of performance, and which have the advantage that they have a hydrophobic group of optionally bio-resourced origin.

### Example 3

This example illustrates the use of copolymers according to the invention and prior art, as thickening agents of a solvent-based satin paint.

The composition of the paint is given in table 3, where the masses of each constituent are given in grams. All the thickening agents have a dry extract of 30% by weight of active matter. The paint is formulated using the methods well known to the skilled man in the art.

TABLE 3

| Ingredients of the water-based paint formulation | Masses (g) |
|---|---|
| Coadis ™ BR3 (COATEX ™ dispersing agent) | 3.5 |
| Tego 1488 ™ (TEGO ™ anti-foaming agent) | 2.0 |
| Mergal K6N (TROY ™ bactericide) | 2.0 |
| TiONa RHD2 (TiO₂ HUNTSMAN ™) | 190 |
| Omyacoat ™ 850 OG (CaCO₃ OMYA ™) | 130 |
| Texanol ™ (EASTMAN CHEMICALS ™ coalescence agent) | 20.0 |
| Acronal ™ 290 D (BASF ™ binder) | 424 |
| NaOH | 5.1 |
| Monoethylene glycol | 20.0 |
| Tested copolymer | 5.0 |
| Water | 198.4 |
| Total | 1000 |

Tests B1, B3, B5 respectively use copolymers 1, 3 and 5 according to the invention (INV) and test B6 uses the same copolymer from the prior art (PA) as for test A6. The results are shown in table 4.

TABLE 4

| | | | test B3 | test B5 | test B6 |
|---|---|---|---|---|---|
| | | test B1 INV | INV | INV | PA |
| μ_{B10}/μ_{B100} | T = 0 | 2840/670 | 4800/1020 | 6480/1270 | 4640/1000 |
| μ_{S} | | 66 | 73 | 78 | 73 |
| μ_{I} | | 0.4 | 0.4 | 0.5 | 0.5 |
| μ_{B10}/μ_{B100} | T = 24 hours | 4520/1000 | 7520/1490 | 12300/2160 | 9760/1830 |
| μ_{S} | | 73 | 82 | 89 | 87 |
| μ_{I} | | 0.4 | 0.4 | 0.5 | 0.5 |
| μ_{B10}/μ_{B100} | T = 7 days | 6300/1270 | 10500/1900 | 13100/2160 | 13100/2100 |
| μ_{S} | | 81 | 90 | 94 | 94 |
| μ_{I} | | 0.4 | 0.4 | 0.5 | 0.5 |

This table demonstrates that the copolymers according to the invention enable a solvent-free matt paint to be thickened effectively, whatever the shearing gradient. Test B5 even enables viscosities to be obtained which are higher than those measured according to the prior art B6, despite using only 2.5% by weight of associative monomer, compared to 10% for the product of the prior art.

Moreover, the variations observed according to the synthesised structures demonstrate the possibility of developing a range of thickening agents from such copolymers. The latter therefore constitute an effective alternative in terms of performance, and which have the advantage that they have a hydrophobic group of bio-resourced origin.

### Example 4

This example illustrates the use of copolymers according to the invention and prior art, as thickening agents of a wood stain.

The composition of the wood stain is indicated in table 5. All the thickening agents have a dry extract of 30% by weight of active matter. The paint is formulated using the methods well known to the skilled man in the art.

TABLE 5

| Ingredients of the water-based paint formulation | Masses (g) |
|---|---|
| Nopco ™ (COGNIS ™ anti-foaming agent) | 1.3 |
| Mergal K6N (TROY ™ bactericide) | 17.1 |
| Acematt TS 100 ™ (DEGUSSA ™ silica) | 2.4 |
| Acronal ™ LR 9014 (BASF ™ binder) | 337.38 |
| Black Luconyl ™ (BASF ™ pigment) | 0.06 |
| Red Luconyl ™ (BASF ™ pigment) | 0.3 |
| Black Luconyl ™ (BASF ™ pigment) | 12.06 |
| NH₄OH | 2.25 |
| Tested copolymer | 5 |
| Water | 222.15 |
| Total | 600 |

Test C3 uses copolymer 3 according to the invention (INV) and test C6 uses the same product of the prior art (PA) as test A6. The results are shown in table 6.

TABLE 6

| | | test C3 | test C6 |
|---|---|---|---|
| | | INV | PA |
| μ_{B10}/μ_{B100} | T = 0 | 3360/870 | 3150/820 |
| μ_{S} | | 69 | 69 |
| μ_{I} | | 0.3 | 0.3 |
| μ_{B10}/μ_{B100} | T = 24 hours | 13700/2680 | 14700/2690 |
| μ_{S} | | 98 | 98 |
| μ_{I} | | 0.4 | 0.4 |

This table demonstrates that the copolymer according to the invention enables a wood stain to be thickened effectively, whatever the shearing gradient. The thickening properties are approximately at the same level as the reference of the prior art.

In addition, 2 pigmentary compatibility tests are made. In practice, if a paint has an insufficient pigmentary compatibility, firstly a drop in the viscosity is observed, and also a low colouring force, requiring a greater quantity of colouring agents in order to obtain a certain level of colour. Too light a colour of paint film is then obtained, which lacks uniformity compared to the reference. This phenomenon may be measured by the use of a spectrophotocolorimeter enabling the trichromatic coordinates to be measured (Huntsmann: L*,a*,b*) and thus the colour of a dry paint film.

A “finger-rubbing test” is also made, known to the skilled man in the art by the term “rub out”. Its consists in applying, without shearing, using an applicator, 150 μm of the paint formulation on a contrast card, i.e. slowly and without stress, and in waiting for 45 seconds and then in applying a shearing by rubbing, with the finger, the still viscous paint film, for 30 seconds in any place. After the film has dried the colorimetric difference between the sheared are (the rubbed area) and the non-sheared area (the area where the film has not been rubbed) determined using a Spectro-Pen spectrocolorimeter, enables it to be evaluated (value of ΔE) whether the tested paint composition does or does not have a satisfactory pigmentary compatibility.

These latter 2 tests are made for a paint to which 5% by weight of a dry pigment which is black Laconyl™ (BASF™) has been added.

TABLE 7

| | | test C3 | test C6 |
|---|---|---|---|
| | | INV | PA |
| μ_{B10}/μ_{B100} | T = 0 | 16200/2680 | 27500/3700 |
| μ_{S} | after introduction of | 94 | 104 |
| μ_{I} | the pigment | 0.4 | 0.4 |
| μ_{B10}/μ_{B100} | T = 24 hours | 4280/1700 | 4800/1400 |
| μ_{S} | after introduction of | 75 | 82 |
| μ_{I} | the pigment | 0.3 | 0.3 |
| ΔE | | 0.8 | 0.8 |
| L* black | | 10.0 | 10.0 |
| background | | | |
| L* white | | 10.0 | 10.0 |
| background | | | |

Although the ΔE and L* values are not affected, the drop in viscosity at a low and medium speed gradient is greater in the context of the prior art, which demonstrates an improved pigmentary compatibility with the invention.

### Example 5

This example illustrates the use of copolymers according to the invention and the prior art, as thickening agents of the same wood stain as that used in example 4.

In this case the thixotropic character of the observed rheological profiles, in the case of a thickening agent of the prior art which is the polymer according to test A6 (or C6), of a thickening agent of the invention which is the polymer according to test C5, and of a reference which is a non-thixotropic thickening agent sold by the company COATEX™ with the name Viscoatex 730, are evaluated.

From an experimental standpoint, the test consists in applying, using a Rheostress™ RS 150 rheometer sold by the company HAAKE™, a shearing cycle corresponding to an increase followed by a reduction of the shearing. A rheogram is then established, which is the graph reflecting the changes in viscosity as a function of shearing. If a product is thixotropic the path followed on the return is substantially below the one followed in the outward stage. The amplitude of the thixotropy is quantified by the area determined by the out and return graphs. Thixotropic properties notably facilitate application of the paints on vertical surfaces.

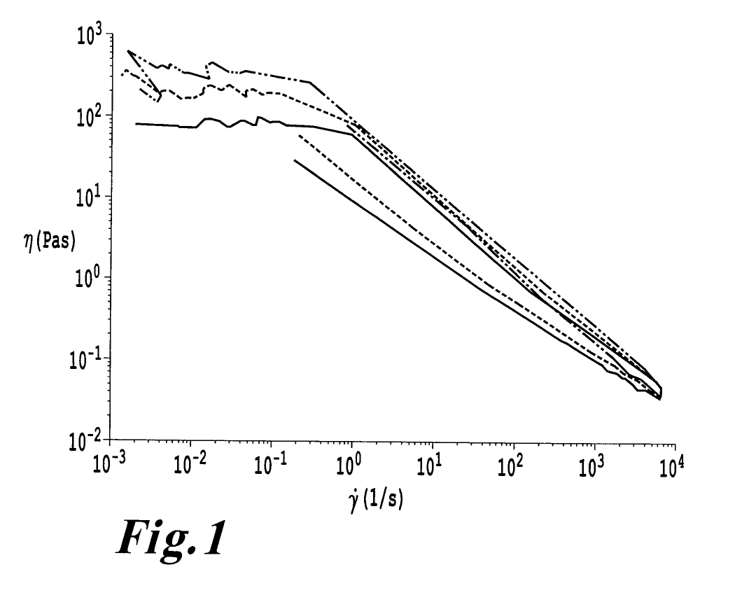

FIG. 1/1 represents the rheograms obtained for a non-thixotropic thickening agent of the prior art which is Viscoatex 730 (shown by the dotted grey line), for the polymer of the prior art A6 (grey line) and for the polymer according to the invention C5 (black line).

The latter has thixotropic properties at least as marked as for the product of the prior art (the hydrophobic ends of which are derived from a fossil energy source): it may therefore be used very advantageously to thicken a paint with a view to an application on a vertical surface.

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. The term “mentioned” notes exemplary embodiments, and is not limiting to certain species. As used herein the words “a” and “an” and the like carry the meaning of “one or more.”

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out. This means for example, that where p is described as a real number of between 24 and 34 that all of 24, 25, 26, 27, 28, 29, 30, 31, 32, 33 and 34 are described and included within the invention and that where a) is described as 20% to 50% that 20, 20.1, 20.2, 20.3, . . . 34.6, 34.65, . . . 47.78 . . . etc are described and included within the invention.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A monomer of formula (I):
R-(A-O)ₘ—(B—O)ₙ—R′ (I)
where:
m and n independently are integers of less than 150, at least one of which is non-zero,
A and B designate alkyl groups which are different one from another, each having 2 to 4 carbon atoms,
R designates a polymerisable unsaturated group, and
R′ is a grouping of formula (II):
CH₃—(CH₂)ₚ—CH₂— (II)
where p is a real number of 24-34.

2. A monomer as claimed in claim 1, wherein AO is ethylene oxide.

3. A monomer as claimed in claim 1, wherein BO is propylene oxide.

4. A monomer as claimed in claim 1, wherein R is methacrylate.

5. A monomer as claimed in claim 1, wherein p is 26.

6. A monomer as claimed in claim 1, wherein AO is ethylene oxide, BO is propylene oxide, R is methacrylate, and p is 26.

7. A hydrosoluble copolymer comprising monomer units derived from:
a) (meth)acrylic acid,
b) at least one ester of (meth)acrylic acid, and
c) at least one monomer of formula (I):
R-(A-O)ₘ—(B—O)ₙ—R′ (I)
where:
m and n independently are integers of less than 150, at least one of which is non-zero,
A and B designate alkyl groups which are different one from another, and having 2 to 4 carbon atoms,
R designates a polymerisable unsaturated group, and
R′ is a grouping of formula (II):
CH₃—(CH₂)ₚ—CH₂— (II)
where p is a real number of 24-34.

8. A hydrosoluble copolymer as claimed in claim 7, wherein AO is ethylene oxide.

9. A hydrosoluble copolymer as claimed in claim 7, wherein BO is propylene oxide.

10. A hydrosoluble copolymer as claimed in claim 7, wherein R is methacrylate.

11. A hydrosoluble copolymer as claimed in claim 7, wherein p is 26.

12. A hydrosoluble copolymer as claimed in claim 7, wherein AO is ethylene oxide, BO is propylene oxide, R is methacrylate, and p is 26.

13. A hydrosoluble copolymer as claimed in claim 7, comprising monomer units derived from a), b), and c) in the following amounts by weight based on total weight:
d) 20% to 50% of (meth)acrylic acid,
e) 40% to 70% of at least one ester of (meth)acrylic acid, and
f) 2% to 20% of at least one monomer of formula (I).

14. A hydrosoluble copolymer as claimed in claim 7, comprising monomer units derived from a), b), and c) in the following amounts by weight based on total weight:
d) 35% to 45% of (meth)acrylic acid,
e) 45% to 55% of at least one ester of (meth)acrylic acid, and
f) 3% to 15% of at least one monomer of formula (I).

15. A hydrosoluble copolymer as claimed in claim 13, where the sum of a), b) and c) is equal to 100%.

16. A monomer as claimed in claim 1, wherein formula (II):
CH₃—(CH₂)ₚ—CH₂— (II)
where p is a real number of 24-34 is obtained from a polycosanyl or a derivative of a polycosanol.

17. A monomer as claimed in claim 1, wherein formula (II) is obtained from a derivative of a polycosanol selected from fatty alcohols derived from sugar cane, beeswax and rice polishings.

18. A monomer as claimed in claim 1, wherein formula (II) is obtained from a polycosanol selected from octacosanol, triacontanol, dotriacontanol, hexacosanol and heptacosanol.

19. A water-based composition comprising a hydrosoluble copolymer as claimed in claim 7 as a thickening agent.

20. A water-based composition as claimed in claim 19, where the composition is a water-based paint, a lacquer, a varnish, a paper coating, a cosmetic, or a detergent formulation.
